SEQUENCE LISTING (9 sequences)

SEQ ID NO: 1 - protein, 697 aa, Homo sapiens
RGSCQACGDSVLVVSEDVNYEPDLDFHQGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 2 - protein, 696 aa, Mus musculus
RGSCQACGDSVLVVDEDVNPEDSGRTTLYWSDLQAMFLRFLGEGRMEDTIRLTSLRERVAGSARMATLVQDILDKLSRQKLDHVWTDTHYVGLQFPDPAHANTLHWVDADGSVQEQLPLEDPEVYCPYSATGNATGKLVYAHYGRSEDLQDLKAKGVELAGSLLLVRVGITSFAQKVAVAQDFGAQGVLIYPDPSDFSQDPHKPGLSSHQAVYGHVHLGTGDPYTPGFPSFNQTQFPPVESSGLPSIPAQPISADIADQLLRKLTGPVAPQEWKGHLSGSPYRLGPGPDLRLVVNNHRVSTPISNIFACIEGFAEPDHYVVIGAQRDAWGPGAAKSAVGTAILLELVRTFSSMVSNGFRPRRSLLFISWDGGDFGSVGATEWLEGYLSVLHLKAVVYVSLDNSVLGDGKFHAKTSPLLVSLIENILKQVDSPNHSGQTLYEQVALTHPSWDAEVIQPLPMDSSAYSFTAFAGVPAVEFSFMEDDRVYPFLHTKEDTYENLHKMLRGRLPAVVQAVAQLAGQLLIRLSHDHLLPLDFGRYGDVVLRHIGNLNEFSGDLKERGLTLQWVYSARGDYIRAAEKLRKEIYSSERNDERLMRMYNVRIMRVEFYFLSQYVSPADSPFRHIFLGQGDHTLGALVDHLRMLRADGSGAASSRLTAGLGFQESRFRRQLALLTWTLQGAANALSGDVWNIDNNF

SEQ ID NO: 3 - protein, 801 aa, Homo sapiens
MERLWGLFQRAQQLSPRSSQTVYQRVEGPRKGHLEEEEEDGEEGAETLAHFCPMELRGPEPLGSRPRQPNLIPWAAAGRRAAPYLVLTALLIFTGAFLLGYVAFRGSCQACGDSVLVVSEDVNYEPDLDFHQGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 4 - protein, 798 aa, Mus musculus
MEQRWGLLRRVQQWSPRPSQTIYRRVEGPQLEHLEEEDREEGAELPAQFCPMELKGPEHLGSCPGRSIPIPWAAAGRKAAPYLVLITLLIFTGAFLLGYVAFRGSCQACGDSVLVVDEDVNPEDSGRTTLYWSDLQAMFLRFLGEGRMEDTIRLTSLRERVAGSARMATLVQDILDKLSRQKLDHVWTDTHYVGLQFPDPAHANTLHWVDADGSVQEQLPLEDPEVYCPYSATGNATGKLVYAHYGRSEDLQDLKAKGVELAGSLLLVRVGITSFAQKVAVAQDFGAQGVLIYPDPSDFSQDPHKPGLSSHQAVYGHVHLGTGDPYTPGFPSFNQTQFPPVESSGLPSIPAQPISADIADQLLRKLTGPVAPQEWKGHLSGSPYRLGPGPDLRLVVNNHRVSTPISNIFACIEGFAEPDHYVVIGAQRDAWGPGAAKSAVGTAILLELVRTFSSMVSNGFRPRRSLLFISWDGGDFGSVGATEWLEGYLSVLHLKAVVYVSLDNSVLGDGKFHAKTSPLLVSLIENILKQVDSPNHSGQTLYEQVALTHPSWDAEVIQPLPMDSSAYSFTAFAGVPAVEFSFMEDDRVYPFLHTKEDTYENLHKMLRGRLPAVVQAVAQLAGQLLIRLSHDHLLPLDFGRYGDVVLRHIGNLNEFSGDLKERGLTLQWVYSARGDYIRAAEKLRKEIYSSERNDERLMRMYNVRIMRVEFYFLSQYVSPADSPFRHIFLGQGDHTLGALVDHLRMLRADGSGAASSRLTAGLGFQESRFRRQLALLTWTLQGAANALSGDVWNIDNNF

SEQ ID NO: 5 - protein, 94 aa, Homo sapiens
YCPYSAIGNVTGELVYAHYGRPEDLQDLRARGVDPVGRLLLVRVGVISFAQKVTNAQDFGAQGVLIYPEPADFSQDPPKPSLSSQQAVYGHVHL

SEQ ID NO: 6 - protein, 186 aa, Homo sapiens
GRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTY

SEQ ID NO: 7 - protein, 126 aa, Homo sapiens
LKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANA

SEQ ID NO: 8 - DNA, 2538 bp, Homo sapiens
  source 1..2538: mol_type = unassigned DNA
gttcgttgcaacaaattgatgagcaatgcttttttataatgccaactttgtacaaaaaagttggcaccatggagcggctttggggtctattccagagagcgcaacaactgtccccaagatcctctcagaccgtctaccagcgtgtggaaggcccccggaaagggcacctggaggaggaagaggaagacggggaggagggggcggagacattggcccacttctgccccatggagctgaggggccctgagcccctgggctctagacccaggcagccaaacctcattccctgggcggcagcaggacggagggctgccccctacctggtcctgacggccctgctgatcttcactggggccttcctactgggctacgtcgccttccgagggtcctgccaggcgtgcggagactctgtgttggtggtcagtgaggatgtcaactatgagcctgacctggatttccaccagggcagactctactggagcgacctccaggccatgttcctgcagttcctgggggaggggcgcctggaggacaccatcaggcaaaccagccttcgggaacgggtggcaggctcggccgggatggccgctctgactcaggacattcgcgcggcgctctcccgccagaagctggaccacgtgtggaccgacacgcactacgtggggctgcaattcccggatccggctcaccccaacaccctgcactgggtcgatgaggccgggaaggtcggagagcagctgccgctggaggaccctgacgtctactgcccctacagcgccatcggcaacgtcacgggagagctggtgtacgcccactacgggcggcccgaagacctgcaggacctgcgggccaggggcgtggatccagtgggccgcctgctgctggtgcgcgtgggggtgatcagcttcgcccagaaggtgaccaatgctcaggacttcggggctcaaggagtgctcatatacccagagccagcggacttctcccaggacccacccaagccaagcctgtccagccagcaggcagtgtatggacatgtgcacctgggaactggagacccctacacacctggcttcccttccttcaatcaaacccagttccctccagttgcatcatcaggccttcccagcatcccagcccagcccatcagtgcagacattgcctcccgcctgctgaggaagctcaaaggccctgtggccccccaagaatggcaggggagcctcctaggctccccttatcacctgggccccgggccacgacttcggctagtggtcaacaatcacaggacctccacccccatcaacaacatcttcggctgcatcgaaggccgctcagagccagatcactacgttgtcatcggggcccagagggatgcatggggcccaggagcagctaaatccgctgtggggacggctatactcctggagctggtgcggaccttttcctccatggtgagcaacggcttccggccccgcagaagtctcctcttcatcagctgggacggtggtgactttggaagcgtgggctccacggagtggctagagggctacctcagcgtgctgcacctcaaagccgtagtgtacgtgagcctggacaacgcagtgctgggggatgacaagtttcatgccaagaccagcccccttctgacaagtctcattgagagtgtcctgaagcaggtggattctcccaaccacagtgggcagactctctatgaacaggtggtgttcaccaatcccagctgggatgctgaggtgatccggcccctacccatggacagcagtgcctattccttcacggcctttgtgggagtccctgccgtcgagttctcctttatggaggacgaccaggcctacccattcctgcacacaaaggaggacacttatgagaacctgcataaggtgctgcaaggccgcctgcccgccgtggcccaggccgtggcccagctcgcagggcagctcctcatccggctcagccacgatcgcctgctgcccctcgacttcggccgctacggggacgtcgtcctcaggcacatcgggaacctcaacgagttctctggggacctcaaggcccgcgggctgaccctgcagtgggtgtactcggcgcggggggactacatccgggcggcggaaaagctgcggccggagatctacagctcggaggagagagacgagcgactgacacgcatgtacaacgtgcgcataatgcgggtggagttctacttcctttcccagtacgtgtcgccagccgactccccgttccgccacatcttcatgggccgtggagaccacacgctgggcgccctgctggaccacctgcggctgctgcgctccaacagctccgggacccccggggccacctcctccactggcttccaggagagccgtttccggcgtcagctagccctgctcacctggacgctgcaaggggcagccaatgcgcttagcggggatgtctggaacattgataacaacttcttgccaactttcttgtacaaagttggcattataagaaagcattgcttatcaatttgttgcaacgaac

SEQ ID NO: 9 - DNA, 3597 bp, Mus musculus
  source 1..3597: mol_type = unassigned DNA
gagtctcctgggagcatggtccaagaaacccagagacctgttgctgagctgaacttggctgctgtgtcttcccactcaggactcggctttgacaggcacgaggcagggactggggtactggtgagcccctacctctcagatctttctggacctggctgcaggtcctggtgtcttcgtcgcggcttggatttcaaactggaggagttcaggagggggcacaagcatggagcaacgttggggtctacttcggagagtgcaacagtggtccccaagaccctctcagaccatctacagacgcgtggaaggccctcagctggagcacctggaggaggaagacagggaggaaggggcggagcttcctgcccagttctgccccatggaactcaaaggccctgagcacttaggctcctgtcccgggaggtcaattcccataccctgggctgcagcaggtcgaaaggctgccccctatctggtcctgatcaccctgctaatcttcactggggccttcctcctaggctacgtggcctttcgagggtcctgccaggcgtgtggggactccgtgttggtggtcgatgaagatgtcaaccctgaggactccggccggaccacgttgtactggagcgacctccaggccatgtttctccggttccttggggaggggcgcatggaagacaccatcaggctgaccagcctccgggaacgcgtggctggctcagccagaatggccaccctggtccaagatatcctcgataagctctcgcgccagaagctggaccacgtgtggactgacacgcactacgtgggacttcagttcccagatccggctcacgctaacaccctgcactgggtggatgcagacgggagcgtccaggagcagctaccgctggaggatccggaagtctactgtccctacagcgccaccggcaacgccacgggcaagctggtgtacgcccactacgggcggtcggaggacctacaggacctaaaagccaagggcgtggagctggccggcagcctcctgctagtgcgagttggaattactagcttcgcccagaaggtagccgttgcccaggactttggggctcaaggagtgctgatataccctgacccatcagacttctcccaggatccccacaagccaggcctgtctagccaccaggctgtgtacggacatgtgcacctgggaactggagacccttacacacctggcttcccgtccttcaatcaaacccagttccctccagtagaatcatcaggccttcccagcatccccgcccagcccatcagtgctgacattgctgatcaattgctcaggaaactcacaggccccgtggctccccaggagtggaaaggtcacctctcaggctctccttatcggctgggacctgggcccgacttacgccttgtggtcaacaaccacagagtctctacccccatcagtaacatctttgcgtgcatcgagggctttgcagagccagatcactatgttgtcattggggcccagagggatgcatggggcccaggagcagccaagtctgcagtggggactgccatcctgctggagctggttcggaccttctcttccatggtcagcaatgggttcagacctcgaagaagtcttttgttcatcagctgggacggaggtgactttggcagcgtgggagccacagagtggttggagggctacctcagcgtgctacacctcaaagctgttgtgtacgtgagcctggacaactccgtgttgggagatggcaaattccatgctaagaccagcccccttctcgtcagcctcattgagaatatcttgaagcaggtggactcccctaaccatagtggacagaccctctatgaacaagtggcactcacccaccccagctgggatgctgaagtgattcagcccctgcccatggacagcagtgcatattccttcacagcctttgcgggggtcccagctgtggagttctccttcatggaggatgatcgggtgtacccattcctgcacacgaaggaggacacatatgagaatctgcacaagatgctgcgaggtcgcctgcccgccgtggtccaggcagtggctcagctcgcgggccagctcctcatccgactgagccacgatcacctactgccgctagacttcggccgctatggagacgtggttctcaggcacatcggcaacctcaatgagttctctggggacctcaaggagcgcgggctgaccctgcagtgggtgtactctgcaaggggggactacatccgtgcggcggaaaagctgcggaaggagatttacagctcggagcggaacgatgagcgtctgatgcgcatgtacaacgtgcgcatcatgagggtggagttctacttcctgtcccagtatgtgtcgccagccgactccccattccgccacattttcctaggccaaggcgaccacactttgggtgccctggtagaccacctgcggatgctgcgcgccgatggctcaggagccgcctcttcccggttgacagcaggtctgggcttccaggagagtcgcttccggcgccagctggcgctgctcacctggacactgcagggggcagccaacgctctcagtggcgacgtttggaacattgacaataacttttgaagccaaaagccctccatgggccccacgtgattctcctttctccctctttgagtggtgcaggcaaaggaggtgcctgagattgtaacctattcttaacacccttggtcctgcaatgctggtgcgccatattttctcagtgtggttgtcatgccgttgcttacccagaaagcggttttcttcccatcacaggcccttctgtcttcaggagcaaagttccccatatctagagactatctagatgctgggatctgatcagctctcttagagagtgagatggacagcgtcattattttatgacacatgagctacggtatgtgagcagcccaaggggattagatgtcaataaaccaattgtaacccctgttgtccatacgcaatttagcttcctcttcatgccgtacccactcctcatatccgccttgagactagggaagaaggcacagaaggcacctgacagcatgcttgcaagatgctatccacatgggaaaaataactgttctgatgtctaagaaaactgcctaaagataatggataggaggtggggcagtggggataactcatcaggaatgggtgattgcggccaagcctgatgacctgagtttaatccccaggaccaacatggcaaaaggagagaactagttcccacaagttttcctatatcctccaaatgtatgcccataaaagcacaaatagataaatgtttttgcttttgttttttaaagtggctttgtggttaagagcactggatgccattctagaagacaccggttcgattcccagcacccacatggccgcttacaactgtctgtaactccagttgctggggatcagatgccctcttctggtatggtgtacgactgcatgcatgtagtatacatacaagcaggcaaaatacccatacatgtaaaatttttaaaaaatagtttaaattataaattaatttaaagaacaaataaaagattaatgcctttaatccc

(19) United States
(12) Patent Application Publication
(10) Pub. No.: US 2024/0197826 A1
(43) Pub. Date: Jun. 20, 2024

(54) USE OF THE EXTRACELLULAR DOMAIN OF TRANSFERRIN RECEPTOR 2 FOR THE DIAGNOSIS AND TREATMENT OF PRIMARY OR SECONDARY SCLEROSING DISEASES

(71) Applicant: Kymab Limited, Cambridge (GB)

(72) Inventors: Martina RAUNER, Dresden (DE); Lorenz C. HOFBAUER, Dresden (DE); Uwe PLATZBECKER, Dresden (DE); Ulrike BASCHANT, Dresden (DE)

(21) Appl. No.: 18/534,366

(22) Filed: Dec. 8, 2023

Related U.S. Application Data
(63) Continuation of application No. 16/622,616, filed on Dec. 13, 2019, now Pat. No. 11,878,048.

(30) Foreign Application Priority Data
Jun. 14, 2017 (EP) 17176043.2

(51) Int. Cl. A61K 38/17; G01N 33/68

(52) U.S. Cl. CPC A61K 38/177; G01N 33/6893

(57) ABSTRACT

The invention relates to a protein for use in diagnosing and treating primary or secondary sclerosing diseases, a fusion protein, and nucleotide sequence and a vector, and to a pharmaceutical composition for use in diagnosing and treating primary or secondary sclerosing diseases.

### CROSS REFERENCE TO RELATED APPLICATIONS

This application is a continuation of U.S. application Ser. No. 16/622,616, filed internationally on Jun. 14, 2018, which is a national stage application under 35 U.S.C. § 371 of International Application No. PCT/EP2018/065846, filed internationally on Jun. 14, 2018, which claims priority benefit to European Application No. 17176043.2, filed Jun. 14, 2017, the contents of which are incorporated herein by reference in their entirety.

### REFERENCE TO AN ELECTRONIC SEQUENCE LISTING

The content of the electronic sequence listing (165062000201 seqlist.xml; Size: 18,301 bytes; and Date of Creation: Dec. 8, 2023) is herein incorporated by reference in its entirety.

### DETAILED DESCRIPTION

The invention relates to a protein for use in diagnosing and treating primary or secondary sclerosing diseases, a fusion protein, and nucleotide sequence and a vector, and to a pharmaceutical composition for use in diagnosing and treating primary or secondary sclerosing diseases.

There are numerous sclerosing diseases that are associated with uncontrolled bone formation, including Fibrodysplasia ossificans progressiva (FOP). This rare disease is characterized by heterotopic ossification (HO) which leads to ossification outside of the skeleton, in particular of muscles, tendons and soft parts, and thus greatly impairs the mobility of patients. FOP sufferers have an average life expectancy of 56 years and the cause of death is often the inability of the thorax to support normal respiration. FOP patients have a mutation in the ACVR1-Gen, which codes for the ACVR1/ALK2 receptor. This receptor is part of the bone morphogenetic protein (BMP) signaling pathway and is of decisive importance in the regulation of cartilage and bone development. This mutation leads to increased activity of the ACVR1/ALK2 receptor and thus to excessive BMP signaling, resulting in increased and uncontrolled bone formation (Shore and Kaplan 2008).

Other sclerosing diseases exist, in addition to FOP, and result from different mechanisms. These include van Buchem disease, sclerosteosis, melorheostosis, pachydermoperiostosis, fibrous dysplasia, osteochondrodysplasia, mucopolysaccharidosis, ankylosing spondylitis, post-traumatic HO, e.g. after joint replacement operations, explosions, amputations, paraplegia, or after calciphylaxis or in the case of malign diseases or degenerative diseases, e.g. prostate carcinomas, renal cell carcinomas, tumoral calcinosis, breast carcinomas, arthrosis or benign bone lesions. These sclerosing diseases are characterized by uncontrolled ossification within and outside of the skeleton.

Conventional methods for treating sclerosing diseases comprise non-specific treatments, steroids, non-steroidal anti-inflammatory drugs (NSAIDs), resection or radiotherapy (Kölbl et al. 2003).

WO 2016/039796 A2 describes a method for treating the sclerosing disease Fibrodysplasia ossificans progressiva (FOP), comprising the administration of activin receptor type 2A (ACVR2A) antagonist and/or activin receptor type 2B (ACVR2B) antagonist or activin receptor type 1 ACVR1) antagonist.

However, the applications of these treatments are limited and usually only symptom-relieving, but cannot prevent the disease from progressing. For example, following resection, the likelihood of HO returning is up to 80%. Steroids in particular cause inhibition of bone formation but have a large number of side effects, such as obesity, diabetes, dry skin or muscle wasting.

WO 2015/107075 A1 discloses the human amino acid sequence of the extracellular domains of transferrin receptor 2 (SEQ ID NO. 1). Baschant et al. describe the iron-dependent, cell-intrinsic negative regulation of osteoclast formation using transferrin receptor 2 (Baschant et al. 2017).

The object of the present invention is therefore that of providing a drug for treating sclerosing diseases.

The object of the invention is furthermore that of providing a drug that has fewer side effects than known treatment methods.

The object is achieved according to the invention by the protein having an amino acid sequence that has at least 70% identity with the sequence of SEQ ID NO. 1, or the fragments thereof, for use in diagnosing and treating primary or secondary sclerosing diseases.

“Identity” is to be understood as the number of matching amino acids based on the total number of amino acids.

A “fragment” is to be understood as a portion of the amino acid sequence of the protein according to the invention, preferably a fragment consisting of the PA domains (SEQ ID NO. 5), a fragment consisting of the peptidase M28 domains (SEQ ID NO. 6), or a fragment consisting of the Tfr-like dimerization domains (SEQ ID NO. 7).

“Primary or secondary sclerosing diseases” are to be understood as diseases associated with ossification of tissue, the sclerosis occurring as a primary or secondary consequence of the disease.

Primary or secondary sclerosing diseases comprise Fibrodysplasia ossificans progressiva (FOP), van Buchem disease, sclerosteosis, melorheostosis, pachydermoperiostosis, fibrous dysplasia, osteochondrodysplasia, mucopolysaccharidosis, ankylosing spondylitis, post-traumatic HO, preferably in the case of scleroses after joint replacement operations, explosions, amputations, paraplegia, calciphylaxis or in the case of malign diseases or degenerative diseases, particularly preferably in the case of prostate carcinomas, renal cell carcinomas, tumoral calcinosis, breast carcinomas, arthrosis and benign bone lesions.

In a preferred embodiment, the use is in diagnosing and treating heterotopic ossification (HO), van Buchem disease, sclerosteosis or Fibrodysplasia ossificans progressiva (FOP).

“HO,” also known as Myositis ossificans, is to be understood as a disease in which ossification of the soft part tissue outside of the skeletal system occurs as a result of tissue injury.

“Fibrodysplasia ossificans progressiva (FOP),” also known
as Fibrodysplasia ossificans multiplex progressiva, Myositis ossificans progressiva or Munchmeyer's disease, is to be understood as a genetic disease in which progressive ossification of the connective and supporting tissue of the human body occurs.

“Van Buchem disease,” also known as van Buchem syndrome, sclerosteosis, Hyperostosis corticalis generalisata familiaris, van Buchem-type endosteal hyperostosis is to be understood as a hereditary skeletal dysplasia comprising hyperplasia of the long bones and the skullcap, which disease is autosomal recessive.

“Fibrous dysplasia” is to be understood as a disease that is caused by a mutation in the GNAS gene and leads to bone excrescences.

“Melorheostosis” is to be understood as a disease that is caused by a mutation in the LEMD3 gene and leads to bone excrescences. This gene codes for a protein that is involved in the transforming growth factor-β (TGF-β) signaling pathway.

“Mucopolysaccharidosis” is to be understood as a group of lysosomal storage diseases that is autosomal recessive and leads to bone changes.

“Ankylosing spondylitis,” Bechterew's disease, Marie-Strumpell disease, ankylosing spondyloarthritis is a chronic inflammatory disease that is preferably manifested in the spinal column and in the sacroiliac joint. In this case, ankylosis and stiffening often occurs in the spinal column.

The protein having an amino acid sequence SEQ ID NO. 1 can be isolated from the human transferrin receptor (Tfr) 2α, the human transferrin receptor (Tfr) 2β, preferably the extracellular domains of human Tfr2α.

The protein according to the invention preferably binds members of
the transforming growth factor-β (TGF-β)/bone morphogenetic proteins BMP families, preferably BMPs, particularly preferably BMP-2, BMP-4, BMP-6 and BMP-7.

“Transforming growth factor-β (TGF-β)/bone morphogenetic protein, BMP families,” is to be understood as a group of similar signaling proteins that bind members of the TGF-β receptor families. The TGF-β/BMP family comprises TGFβ1, TGFβ2, TGFβ3, BMPs, growth differentiation factors (GDFs), activin and inhibin, myostatin, anti-Müllerian hormone (AMH) and nodal.

“Bone morphogenetic proteins (BMPs)” are to be understood as a group of paracrine signaling proteins that bind BMP receptors. In an embodiment, BMPs are selected from BMP1, BMP2, BMP3, BMP4, BMP5, BMP6, BMP7, BMP8a, BMP8b, BMP-9, BMP10 or BMP15, preferably BMP-2, BMP-4, BMP-6 or BMP-7.

Advantageously, when treating primary or secondary sclerosing diseases using the protein according to the invention, the BMP signaling pathway, and thus the bone formation and the Wnt signaling pathway is specifically inhibited.

In an embodiment, the diagnosis of primary or secondary sclerosing diseases using the protein according to the invention is carried out by detecting members of the TGF-β/BMP family, preferably BMPs, particularly preferably BMP-2, BMP-4, BMP-6 and BMP-7.

In an embodiment, the diagnosis of primary or secondary sclerosing diseases using the protein according to the invention is carried out in the blood, in the blood plasma, in the blood serum or in the tissue. In a preferred embodiment, the tissue is bone or cartilage. “Blood plasma” is to be understood as the fluid component of the blood. “Blood serum” is to be understood as the blood plasma without the clotting factor.

In an embodiment, the diagnosis of primary or secondary sclerosing diseases using the protein according to the invention is carried out by means of an immunoassay. An “immunoassay” is to be understood as a detection procedure in which an analyte is detected in a fluid phase by means of antigen-antibody binding.

In an embodiment, the immunoassay is selected from an Enzyme-linked Immunosorbent Assay (ELISA) or Enzyme-linked Immuno Spot Assay (ELIspot Assay). An “ELISA” is to be understood as an antibody-based detection procedure that is based on an enzymatic color reaction. An “ELIspot Assay” is to be understood as a detection procedure for detecting antibodies that are secreted by immune cells following stimulation using antigens and are immobilized on a membrane.

In an embodiment, the diagnosis of primary or secondary sclerosing diseases is carried out in order to assess the prognosis of the disease, to assess the response to treatment and/or for risk stratification. “Risk stratification” is to be understood as assessing the risk of a disease progressing or leading to complications or death.

In an embodiment, the protein according to the invention for use in diagnosing and treating primary or secondary sclerosing diseases comprises sequence SEQ ID NO. 1 or SEQ ID NO. 2.

The protein having an amino acid sequence SEQ ID NO. 2 can be isolated from the murine transferrin receptor (Tfr) 2α, the murine transferrin receptor (Tfr) 2β, preferably the extracellular domains of murine Tfr2α.

In an embodiment, the protein according to the invention comprises 232 amino acids to 801 amino acids, preferably 487 amino acids to 801 amino acids, particularly preferably 600 amino acids to 750 amino acids.

In an embodiment, the protein according to the invention for use in diagnosing and treating primary or secondary sclerosing diseases is the human transferrin receptor (Tfr) 2α (SEQ ID NO. 3), the murine transferrin receptor (Tfr) 2α (SEQ ID NO. 4), the human transferrin receptor (Tfr) 2β (SEQ ID NO. 1) or the extracellular domains of human Tfr2α (SEQ ID NO. 1), the murine transferrin receptor (Tfr) 2β (SEQ ID NO. 2) or the extracellular domains of murine Tfr2α (SEQ ID NO. 2).

The invention further relates to a fusion protein comprising at least one protein according to the invention for use in diagnosing and treating primary or secondary sclerosing diseases.

In an embodiment, the fusion protein comprises at least one protein tag. In an embodiment, the at least one protein tag is selected from a polyhistidine (His) tag, glutathione S-transferase (GST) tag, maltose binding protein (MBP) tag, Myc tag, streptavidin (Strep) tag or a dye, preferably a fluorescent dye, particularly preferably a green fluorescent protein (GFP) or a yellow fluorescent protein (YFP).

In an embodiment, the protein according to the invention or the fusion protein comprising at least one protein according to the invention comprises at least one modification.

In an embodiment, the at least one modification is selected from proteins containing D-amino acids, pseudopeptide bonds, amino alcohols, non-proteinogenic amino acids, amino acids having modified side groups and/or circular proteins.

Proteins comprising modifications are advantageously more stable.

In an embodiment, the protein according to the invention or the fusion protein comprising at least one protein according to the invention is used in the treatment of diseases associated with increased BMP receptor activation.

The invention further relates to a nucleotide sequence comprising a sequence coding for a protein according to the invention or a fusion protein comprising at least one protein according to the invention for use in diagnosing and treating primary or secondary sclerosing diseases.

In an embodiment, the nucleotide sequence comprises SEQ ID NO. 8 or SEQ ID NO. 9.

A further aspect of the invention relates to a vector for use in diagnosing and treating primary or secondary sclerosing diseases, comprising a nucleotide sequence comprising a sequence coding for a protein according to the invention or a fusion protein comprising at least one protein according to the invention.

A “vector” is to be understood as a nucleic acid carrier for transferring a nucleic acid into a cell by means of transfection or transduction. In an embodiment, vectors are selected from plasmids, viral vectors or other nucleic acid carriers that contain a nucleotide sequence comprising a sequence coding for a protein according to the invention or a fusion protein comprising at least one protein according to the invention by means of genetic recombination (recombinant).

The invention further relates to a pharmaceutical composition for use in treating primary or secondary sclerosing diseases, comprising at least one protein according to the invention or a fusion protein comprising at least one protein according to the invention.

In an embodiment, the pharmaceutical composition is a solution, tablet or capsule. In an embodiment, the protein according to the invention is used as a coating for implant materials, preferably metals or plastics materials, and/or implants, preferably protheses, screws or nails.

In an embodiment, the pharmaceutical composition is administered locally in an intraarticular or intramuscular manner, or systemically in a subcutaneous or intravenous manner, or by means of oral administration. In an embodiment, the pharmaceutical composition is in a suitable form for intraarticular, intramuscular, subcutaneous, intravenous or oral administration.

In an embodiment, the pharmaceutical composition contains the protein according to the invention or the fusion protein comprising at least one protein according to the invention in a dose of from 10 μg/kg to 100 mg/kg body weight per administration.

In a further embodiment, the pharmaceutical composition furthermore contains a pharmaceutically acceptable diluent or base. In an embodiment, the pharmaceutically acceptable diluent or base is an aqueous solution, preferably a buffered aqueous solution, an aqueous saline solution or an aqueous glycine solution. In an embodiment, the buffered aqueous solution is selected from a histidine-buffered aqueous solution having a pH of from pH 5.0 to pH 7.0, or a sodium succinate-, sodium citrate-, sodium phosphate-, or potassium phosphate-buffered aqueous solution. In an embodiment, the buffered aqueous solution has a concentration of from 1 mmol/l (mM) to 500 mM, preferably 1 mM to 50 mM. In a further embodiment, the pharmaceutically acceptable diluent or base comprises sodium chloride, preferably in a concentration of between 0 mM and 300 mM, particularly preferably in a concentration of 150 mM.

In an embodiment, the pharmaceutical composition furthermore comprises at least one pharmaceutically acceptable excipient. An “excipient” is understood to be a compound that adjusts physiological conditions with regard to the pH and/or the ionic strength, and/or increases the stability of the pharmaceutical composition. In an embodiment, the at least one pharmaceutically acceptable excipient is selected from sodium acetate, sodium chloride, potassium chloride, calcium chloride or sodium lactate.

In an embodiment, the pharmaceutical composition is sterile. The pharmaceutical composition is sterilized by means of known methods.

A further aspect of the invention relates to the use of the pharmaceutical composition in diagnosing and treating primary or secondary sclerosing diseases.

In an embodiment, the use of the pharmaceutical composition is for administration to a subject. A “subject” is to be understood as an individual or a patient. In an embodiment, the subject is selected from humans or animals. In an embodiment, are selected from rodents, preferably mice, rats, hamsters or guinea pigs; dogs, rabbits, farm animals, preferably goats, sheep, pigs; and non-human primates, preferably chimpanzees, orangutans or gorillas.

In an embodiment, the pharmaceutical composition is used in diagnosing members of the TGF-β/BMP family and treating diseases associated with increased BMP receptor activation.

The invention further relates to a method for diagnosing and/or treating primary or secondary sclerosing diseases, comprising administering the protein according to the invention and/or the pharmaceutical composition.

In an embodiment, the diagnosis and/or treatment of primary or secondary sclerosing diseases is carried out on humans.

For the diagnosis and/or treatment, a sterile pharmaceutical composition, containing a pharmacologically active dose of one or more proteins according to the invention, is administered to a patient in order to diagnose and/or treat primary or secondary sclerosing diseases.

In an embodiment, the administration takes place locally, preferably as a intraarticular or intramuscular injection; or systemically, preferably as a subcutaneous, intramuscular or intravenous injection or infusion, or by means of oral or transdermal administration.

A further aspect of the invention relates to the use of the protein according to the invention or the fusion protein comprising at least one protein according to the invention in diagnosing members of the TGF-β/BMP family or in diagnosing diseases associated with increased BMP receptor activation.

“Increased BMP receptor activation” is to be understood as activation of at least one BMP receptor, which activation is brought about by a mutation of a BMP receptor (Shore and Kaplan 2008), preferably constitutively activating mutations. “Constitutively activating mutations” are to be understood as mutations in which at least one BMP receptor is activated in the absence of BMPs.

A further aspect of the invention relates to the use of a nucleotide sequence, comprising a sequence coding for a protein according to the invention or a fusion protein comprising at least one protein according to the invention, and/or of a vector, comprising a nucleotide sequence comprising a sequence coding for a protein according to the invention or a fusion protein comprising at least one protein according to the invention, in diagnosing members of the TGF-β/BMP family or in diagnosing diseases associated with increased BMP receptor activation.

In addition to the use in diagnosing and/or treating primary or secondary sclerosing diseases, the proteins according to the invention are suitable for biological research and other applications in which the detection of a member of the TGF-β/BMP family is of interest. Applications of this kind are in particular Western Blot, immunostaining of cells (e.g. for flow cytometry and microscopy) and ELISA, and the use as a tracer in imaging techniques such as CT (computer tomography), PET/CT (positron emission tomography).

In order to implement the invention, it is also expedient to combine the above embodiments and features of the claims.

### EMBODIMENTS

The invention will be explained in greater detail in the following, with reference to some embodiments and accompanying drawings. In this case, the embodiments are intended to describe the invention but without having a limiting effect.

### In the Drawings:

FIG. 1: schematic depiction of the influence of BMPs (1) on bone formation (left-hand side). BMPs bind BMP receptors (BMPR-I (2) or BMPR-II (3)), triggering a signaling cascade (phosphorylation (8) of Smad protein (6) and MAP kinase (7)) which activates bone formation (10) by the expression of osteoblast genes (9). Schematic depiction of the binding of BMPs (1) to Tfr2α (4) (center). Schematic depiction of the influence of the protein (5) according to the invention on bone formation (10) (right-hand side). The proteins (5) according to the invention bind BMPs (1), as a result of which binding to BMP receptors (BMPR-I (2) or BMPR-II (3)) does not occur, and bone formation is not activated.

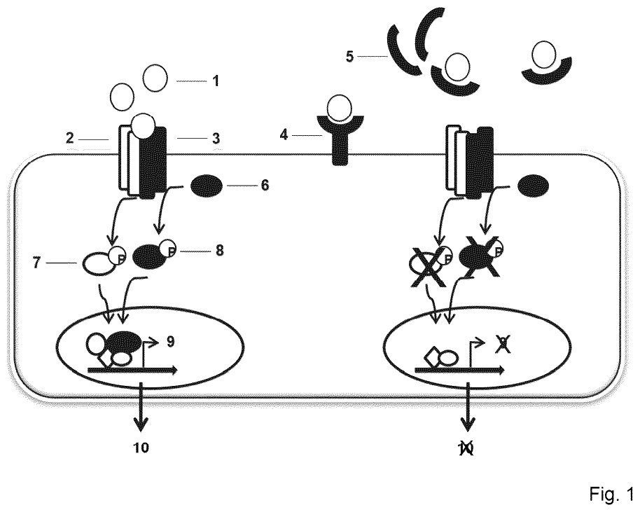

FIGS. 2A-2B show SPR measurements of the binding of BMPs and the proteins according to the invention. FIG. 2A shows the binding of BMP-2, BMP-4, BMP-6 and BMP-7 to Tfr2-ECD. FIG. 2B shows a quantification of the binding level based on the molar mass of BMP-2, BMP-4, BMP-6 and BMP-7 to Tfr2-ECD compared with the binding level of BMPR-II and BMPR-IA.

FIG. 3A schematically shows the BMP-2-competitive ELISA (enzyme linked immunosorbent assay): signal due to binding of BMPs (1), in particular BMP-2, to the capture antibody (11) and binding of the detection antibody (12) to BMP-2 (left-hand side). Reduced signal owing to the binding of the protein (5) according to the invention or BMPR-I (2) to BMP-2, as a result of which binding of the capture antibody (11) and detection antibody (12) does not occur (right-hand side). FIG. 3B: BMP-2-competitive ELISA: Influence of the concentration of the protein according to the invention or BMP-I on the signal of the BMP-2 detection antibody while the BMP-2 concentration remains unchanged.

FIGS. 4A-4B show the inhibition of HO in mice (C57BL/6 mice) using the protein according to the invention, in particular Tfr2-ECD, by means of binding of BMP-2. FIGS. 4A and 4B show the mineralization by determining the bone volume by means of μCT (microtomography). FIG. 4A shows the CT scans of the bone formation when BMP-2 is applied and when BMP-2 is applied together with Tfr2-ECD. FIG. 4B shows the quantification of the bone volume. PBS is used as a negative control. When BMP-2 is applied, the increase in the bone volume is evident after two weeks. Applying BMP-2 together with Tfr2-ECD exhibits a significant reduction in bone formation compared with BMP-2 as a reference (average value±standard deviation; n=3-6 per group; *** p<0.001 with respect to PBS (control).

### Preparation of the Tfr2 Extracellular Domains (Tfr2-ECD)

The nucleic acid sequence of the entirety of the murine extracellular domains (ECD, aa 103-798) of Tfr2, including a 6× His tag, is carried out by Genscript (Germany). The recombinant His-Tfr2-ECD is expressed in Sf9 insect cells using the baculovirus expression system (pOCC211-Tfr2-ECD). Cell culture supernatants are collected and purified using a HisTrap column. After the step of washing using phosphate-buffered saline solution (PBS), the His-Tfr2-ECD-protein is eluted with imidazole by means of PBS.

### Surface Plasmon Resonance Measurement (SPR)

The interactions between Tfr2-ECD and BMPs (BMP-2, −4, −6, −7 by R&D Systems) and BMP receptors (BMPR-IA, BMPR-II by R&D Systems) are analyzed using Biacore T100 (GE Healthcare).

For this purpose, Tfr2-ECD is immobilized on a Series S Sensor Chip C1 (GE Healthcare), by means of coupling the amino groups at 25° C. The carboxyl groups on the chip surface are activated for 7 minutes using a mixture of 196 mM 1-ethyl-3-(3-dimethylaminopropyl)-carbodiimide-hydrochloride and 50 mM n-hydroxysuccinimide at a flow rate of 10 μl/min. Subsequently, 5 μg/ml Tfr2-ECD, diluted with sodium acetate buffer (pH 4.5), is injected at a flow rate of 5 μl/min up to a relative occupancy of 200 RU. Non-reacted groups are inactivated by injecting 1 M thanolamine-HCl (pH 8.5) for 7 minutes at a flow rate of 10 μl/min. In order to prepare a reference surface, the same procedure is carried out but without Tfr2-ECD being injected.

The binding assays are carried out at 37° C. at a flow rate of 30 μl/min. Each analyte is diluted by a running buffer (HBS-P, pH 7.4 together with 50 nM FeCl₃). BMPs are used in a concentration of 50 nM and BMP receptors are used in a concentration of 200 nM. The binding assays are carried out by means of analyte injection for 300 s over the Tfr2-ECD surface, followed by dissociation for 1000 s. The values of the binding level are read out, relative to the baseline, 10 s before the end of the injection and are corrected with respect to the molar mass. Following the dissociation for 1000 s, the chip surface is regenerated for 60 s by means of HBS-P together with 5 M NaCl and 50 mM NaOH and is stabilized for 1000 s. The binding parameters are determined using the Biacore™ T100 evaluation software 2.03.

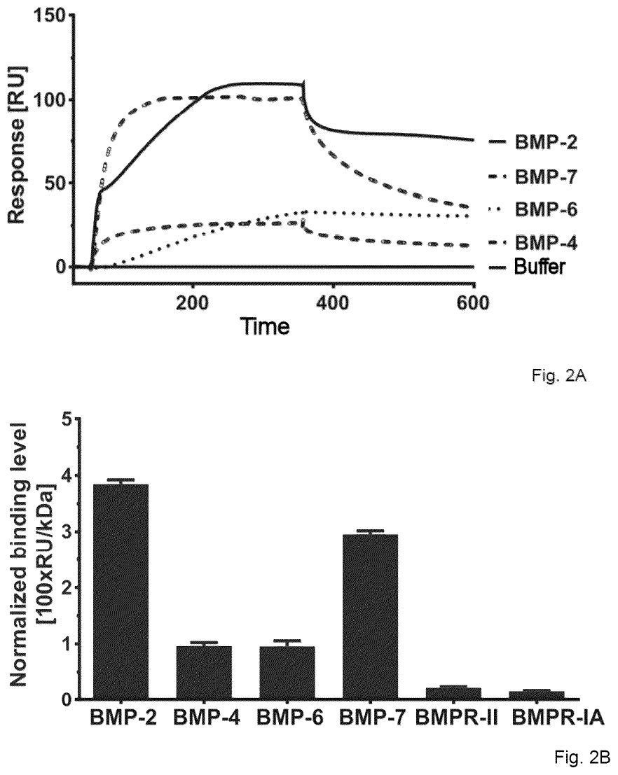

FIG. 2 shows the binding of the protein according to the invention to various BMP ligands (BMP-2, BMP-4, BMP-6, BMP-7).

### Bmp-2-Competitive ELISA (Enzyme Linked Immunosorbent Assay)

In order to carry out the BMP-2-competitive ELISA, the Duo Set BMP-2 ELISA kit by R&D Systems is used. After the plate has been coated, overnight, with BMP-2 capture antibodies, 1.5 ng/ml BMP-2 together with increasing concentrations of Tfr2-ECD or BMPR-IA (positive control, R&D Systems) is added to the assay. Following incubation for 1 hour at room temperature and intensive washing, the detection antibody is added according to the manufacturer's specifications and the amount of BMP-2 not bound to Tfr2-ECD or BMPR-IA that is bound to the capture and detection antibody is quantified.

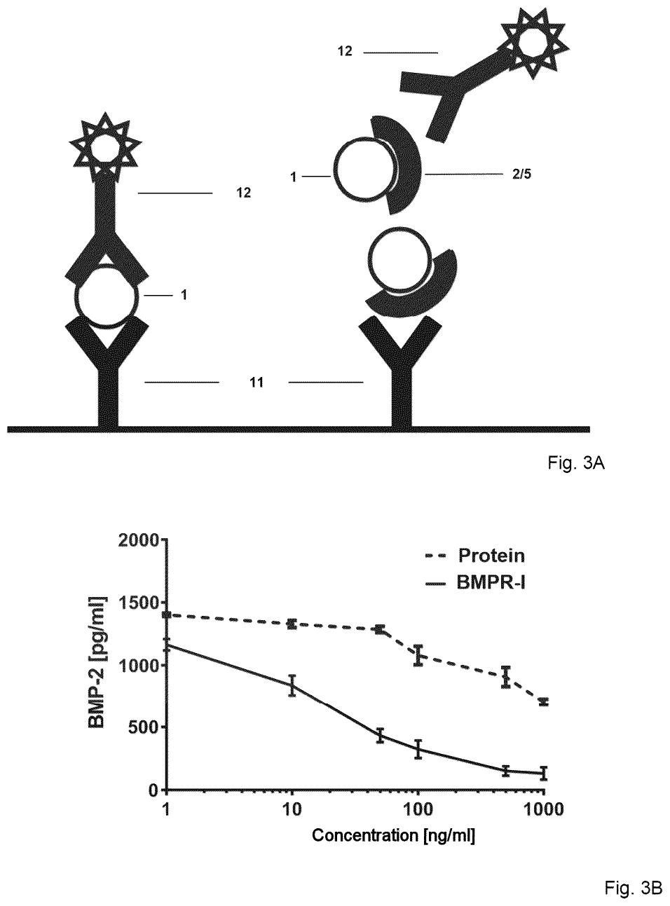

FIG. 3 shows the binding of the protein according to the invention to BMPs, in particular BMP-2. As the concentration of the protein according to the invention increases, the signal of the BMP-2 detection antibody reduces, despite the BMP-2 concentration remaining the same. The progression is comparable to the binding of BMP-2 to the BMP receptor I (BMPR-I).

### Mouse Model of Heterotopic Ossification (HO)

Male and female C57BL/6 mice are used for the HO model. The HO is triggered by injecting BMP-2 into the muscle (Wosczyna et al. 2012).

All the mice are fed with standard feed and water ad libitum and are kept in groups of five mice per cage. The mice are exposed to a 12-hour light/dark cycle and air cooling to 23° C. (no special pathogen-free room). Enrichment is provided in the form of cardboard houses and bedding material. The mice are randomly divided into the different treatment groups and the assays are subsequently carried out as blind experiments.

The HO is examined by means of treatment using 2.5 μl of a 1 mg/ml recombinant BMP-2 solution (Thermo Fisher Scientific) or 2.5 μl of a 1 mg/ml Tfr2-ECD mixed with 47.5 μl Matrigel (BD Bioscience) at 0° C. For combination treatment, 2.5 of a 1 mg/ml recombinant BMP-2 solution is mixed with 2.5 μl of a 1 mg/ml Tfr2-ECD and 45 μl Matrigel. The Matrigel mixtures are injected into the musculus tibialis anterior of 10-week-old female wild-type mice. The legs are examined after two weeks.

### μCT (Microtomography) and Bone Micromineralization Density

The bone microarchitecture is analyzed using vivaCT40 (Scanco Medical, Switzerland). The entire lower leg bones are measured at a resolution of 10.5 μm using X-rays of 70 kVp, 114 mA and at an integration time of 200 s. Predefined scripts from Scanco are used to analyze the bone (#1).

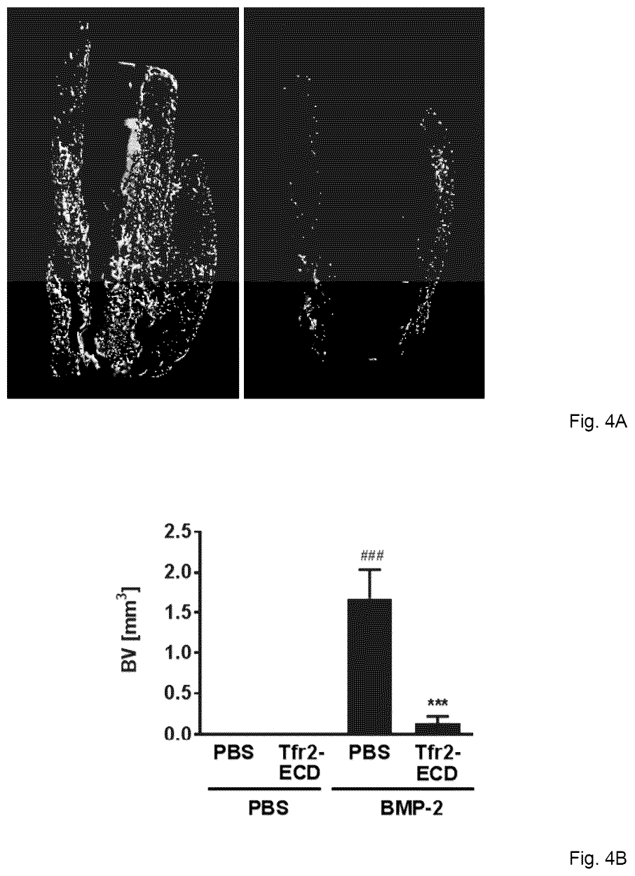

The mouse model of HO develops a BMP-2-induced ossification of the muscle tissue. FIG. 4 shows the inhibition of the ossification or HO in mice (C57BL/6 mice) by Tfr2-ECD, by means of binding of BMP-2.

### Statistical Analysis

The data are specified as the average value±standard deviation (SD). Graphs and statistics are created using Graphpad Prism 6.0-Software. The normality of the data is determined by means of the Kolmogorow-Smirnov Test. In the case of normal distribution, statistical evaluations are carried out by means of two-sample comparison, using the Student's T-Test two-sample test. A one-way analysis of variance (ANOVA) is used for experiments having more than two groups. A two-way ANOVA comprising a Bonferroni post hoc test is used to analyze the treatment effects. If data do not correspond to the normal distribution, the Mann-Whitney U test and the Wilcoxon signed-rank test are used for the data analysis.

### Non-Patent Literature Cited

Shore E M, Kaplan F S (2008) Insights from a rare genetic disorder of extra-skeletal bone formation, fibrodysplasia ossificans progressiva(FOP). Bone 43: 427-433.

Kölbl O, Barthel T, Krödel A, Seegenschmiedt M H (2003) Prävention von heterotopen Ossifikationen nach Totalendoprothese des Hüftgelenks [Prevention of heterotopic ossification following total replacement of the hip joint]. Deutsches Ärzteblatt [German medical journal] 45: 2944-2954. Baschant U, Sastre E A, Roetto A, Platzbecker U, Hofbauer L C, Rauner M (2017) P-OCBR-7: The iron-sensing receptor Tfr2 regulates osteoclastogenesis. Abstracts of the ECTS congress 2017. ECTS 2017. 13-16 May 2017, Salzburg, Austria. 44th European Calcified Tissue Society Congress: Page 43.

Roetto, A. et al. Comparison of 3 Tfr2-deficient murine models suggests distinct functions for Tfr2-alpha and Tfr2-beta isoforms in different tissues. Blood 115, 3382-3389, doi: 10.1182/blood-2009-09-240960 (2010).

Wosczyna M W, Biswas A A, Cogswell C A, and Goldhamer D J (2012) Multipotent Progenitors Resident in the Skeletal Muscle Interstitium Exhibit Robust BMP-Dependent Osteogenic Activity and Mediate Heterotopic Ossification. J Bone Miner Res 27: 1004-1017.

### LIST OF REFERENCE SIGNS

1 BMP
2 BMP-I
3 BMP-II
4 Tfr2α
5 protein
6 Smad protein
7 MAP kinase
8 phosphorylation
9 osteoblast genes
10 bone formation
11 capture antibody
12 detection antibody

1. A method of treating a primary or secondary sclerosing disease in a subject, comprising administering to the subject a protein comprising an amino acid sequence that has at least 70% identity with the sequence of SEQ ID NO: 1, or a fragment thereof.

2. The method of claim 1, wherein the protein comprises sequence SEQ ID NO: 1 or SEQ ID NO: 2.

3. The method of claim 1, wherein the protein has a length of from 232 amino acids to 801 amino acids.

4. The method of claim 1, wherein the protein comprises a transferrin receptor (Tfr) 2α, a transferrin receptor (Tfr) 2β or an extracellular domain of Tfr2α.

5. The method of claim 1, wherein the protein comprises the human transferrin receptor (Tfr) 2α (SEQ ID NO: 3), the murine transferrin receptor (Tfr) 2α (SEQ ID NO: 4), the human transferrin receptor (Tfr) 2β (SEQ ID NO: 1), the extracellular domains of human Tfr2α (SEQ ID NO: 1), the murine transferrin receptor (Tfr) 2β (SEQ ID NO: 2) or the extracellular domains of murine Tfr2α (SEQ ID NO: 2).

6. The method of claim 1, wherein the protein is a fusion protein.

7. The method of claim 1, wherein the protein comprises at least one modification selected from the group consisting of proteins containing D-amino acids, pseudopeptide bonds, amino alcohols, non-proteinogenic amino acids, amino acids having modified side groups, circular proteins, and combinations thereof.

8. The method of claim 1, wherein the disease is associated with increased Bone Morphogenetic Protein (BMP) receptor activation.

9. (canceled)

10. (canceled)

11. The method of claim 1, wherein the protein is present in a pharmaceutical composition.

12. The method of claim 8, wherein the protein is present in a pharmaceutical composition.

13. A method of diagnosing a disease associated with increased Bone Morphogenetic Protein (BMP) receptor activation in a subject, comprising detecting a member of the TGF-β/BMP family using a protein comprising an amino acid sequence that has at least 70% identity with the sequence of SEQ ID NO: 1 in a subject.

14. (canceled)

15. A method of diagnosing a primary or secondary sclerosing disease in a subject, comprising detecting a member of the TGF-β/BMP family using a protein comprising an amino acid sequence that has at least 70% identity with the sequence of SEQ ID NO: 1 in a subject.
